(19) United States
(12) Patent Application Publication
(10) Pub. No.: US 2009/0041743 A1
(43) Pub. Date: Feb. 12, 2009

(54) SPERM-BLOCKING AGENT COMPOSITION WITH CONTRACEPTIVE EFFECT

(76) Inventor: Ma Te Lun, Kaohsiung Hsien (TW)

Correspondence Address: LEONG C LEI, PMB # 1008, 1867 YGNACIO VALLEY ROAD, WALNUT CREEK, CA, 94598, US

(21) Appl. No.: 11/836,807

(22) Filed: Aug. 10, 2007

(51) Int. Cl. A61K 31/715; A61K 31/718; A61K 31/723; A61K 31/724; A61K 31/732; A61K 38/46; A61P 15/18

(52) U.S. Cl. 424/94.6; 514/54; 514/57; 514/58; 514/60

(57) ABSTRACT

A sperm-blocking agent is disclosed. The composition comprises an aqueous solvent a gum which is a polysaccharide having about 0.3 to 10 wt % of the composition, wherein the polysaccharide is selected from the group consisting of celluloses and their derivatives, hemi-cellulose, fiber, starches and their derivatives, polydextrans and their derivatives, pectins and their derivatives, seaweed polysaccharids, their salts and derivatives and natural gums; and a lubricant being alcohols having about 0.5 to wt % of the composition, wherein the alcohol is at least one selected from polyethene glycol having a molecular weight of over 200, polypropylene glycol having a molecular weight poly(ethylene oxide) polymers and glycerin; wherein the viscosity of the composition is above 9000 cst.

### BACKGROUND OF THE INVENTION

(a) Technical Field of the Invention

The present invention relates to sperm-blocking agent composition with contraceptive effect.

(b) Description of the Prior Art

Common contraceptive methods that available include using of condoms made from latex or the application of foam spermicide agents. Condoms are generally made from latex material and are leakage-proof which could block sperms from reaching the uterus of the female during sexual intercourse. However, these condoms may be not all the time leakage proof due to leakage; this will cause a failure in contraception. Further, leakage in condom is generally not visible to our naked eyes. Moreover, to some people, using a condom may affect the climax of sexual intercourse. Accordingly, in view of the drawbacks, application of condoms as a mean of contraception is not effective to certain extent and is not popular.

Foam spermicide having a foaming agent as a base is employed in killing sperms ejected from the male. However, the foaming agent of the spermicide may cause allergic to some people. Moreover long period of application of foam spermicide may cause a deformation to membrane tissues. In addition, foam spermicide contained in a pressurized container is not conveniently to be carried along while traveling. Another drawback of using the foam spermicide is that after a long period of application, the foams will decrease and effectiveness of foam to isolate sperms is greatly reduced.

### SUMMARY OF THE INVENTION

The primary purpose of the present invention is to provide a sperm-blocking agent composition with contraceptive effect, which could block sperms from reaching the uterus of a female, and thus provides a contraceptive effect.

Yet still another object of the present invention is to provide a sperm-blocking agent composition with contraceptive effect comprising: (a) an aqueous solvent; (b) a gum which is a polysaccharide having about 0.3 to 10 wt % of the composition, wherein the polysaccharide is selected from the group consisting of celluloses and their derivatives, hemi-cellulose, fiber, starches and their derivatives, polydextrans and their derivatives, pectins and their derivatives, seaweed polysaccharids, their salts and derivatives and natural gums; and (c) a lubricant which contains alcohols having about 0.5 to wt % of the composition, wherein the alcohol is at least one selected from polyethene glycol having a molecular weight of over 200, polypropylene glycol having a molecular weight of over 200, poly(ethylene oxide) polymers and glycerin; wherein the viscosity of the composition is above 9000 cst.

Still a further object of the present invention is to provide a sperm-blocking agent composition with contraceptive effect, wherein the agent forms into a high viscous gel when it mixes with sperm and fluid secretion from vagina, the gel blocking the cervical preventing the sperm from reaching the uterus. The agent being used can be cleaned away with water.

A further object of the present invention is to provide a sperm-blocking agent composition with contraceptive effect, wherein the packages of the sperm-blocking agent are convenient carried along during traveling.

A further object of the present invention is to provide a sperm-blocking agent (SBA) composition with contraceptive effect, wherein the sperm-blocking agent does not affect the climax of sexual intercourse, and no special care is needed with respect of application. Thus, such a sperm-blocking agent shall be easily accepted by the public. Further, a female could apply the agent herself without causing impotence to a male by putting on a condom onto the penis.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

In accordance with the present invention, the sperm-blocking agent composition contains an aqueous solvent, a gum and a lubricant. The aqueous solvent in the preferred embodiment of the present invention is pure water.

Gum in the preferred embodiment of the present invention is of appropriate viscosity, which is above 9000 cst, and gum is polysaccharids about 0.3-10 wt % of the total composition. The polysaccharids is selected from one of the followings: celluloses and their derivatives, hemi-cellulose, fiber, starches and their derivatives, polydextrans and their derivatives, pectins and their derivatives, seaweed polysaccharids, their salts and derivatives and natural gums.

The celluloses and their derivatives are selected from one of the following: carboxymethyl celluloses, and their derivatives, carboxyethyl celluloses and their derivatives, hydroxypropyl methyl celluloses (HPMC), and their derivatives, methyl celluloses (MC), and their derivatives, hydroxypropyl cellulose (HPC), and their derivatives, hydroxyethyl celluloses (HEC) and their derivatives, methyl hydroxyethyl celluloses (MHEC) and their derivatives, hydroxyl propyl methyl cellulose phthalates (HPMCP), and their derivatives.

The starches and their derivatives are amylase and amylopectine and products of depolymerisation or derivatision crosslinking, and can be selected from α-cyclodextrin amylases and their derivatives, β-cyclodextrin amylases and their derivatives, δ-cyclodextrin amylases and their derivatives, ζ-cyclodextrin amylases and their derivatives, ethylamide from starches and anhydrides, dially ether from reaction of starches and epoxy methane, and reactant of starches and α-octyl butyl di-ester.

Petins and their derivatives are selected from at least one of the material: L-Rhamnose, high methoxyl pectin and low methoxyl pectin with a main chain or brach chain provided with a long chain.

Seaweed polysaccharids, their salt and derivatives are selected from at least one of the following: pheophycase, β, D-mannuroic acid, and α-L-guluronic (G) which is arranged in M-M-M-M-M, -G-G-G-G-G or M-G-M-G-M-G; agarose, λ-carageenan, 1-carageenan, sodium alginiate, chondrus, Eucheuma, Gigartina, and hypnea.

Natural gum is selected from one of the following: xanthan gum, konjac gum, agar, Flaxseed gum, arabic gum, guar gum, aloe Vera gel, gellan gum, locust bean gum, curdlan gum, Tara gum, guar gum, Tragant gum, Tamarind gum, Karaya gum, and Furcellaran.

In the present preferred embodiment, gum additive is gelatin, silicone gel, sodium polyacrylates and their derivatives.

The lubricant contains alcohol which is about 0.5 to 10 wt % of the composition. Alcohol is selected from one of the following:
polyethene glycol of molecular weight of more than 200;
polypropylene glycol of molecular weight of more than 200, high molecular weight poly(ethylene oxide) polymers and glycerin.

In accordance with the present invention, the lubricant additive is polymer of arylamide and dimethy (dialy) ammonium salt.

The composition, in the preferred embodiment, further comprises a salt additive, which is sodium chloride.

In accordance with the present invention, the composition is an aqueous high viscosity liquid/gel. In application, the composition is injected into the vagina of a female. During intercourse, the penis of a male will push the composition into the bottom portion of the vagina. When sperm of the male is mixed with the composition of the present invention, the water content of the fluid secreted from the vagina and sperm will be absorbed, and thus the sperm shall not be moving around easily and the gel formed from the mixing of the composition and the fluid block the cervical of the vagina. Thus, sperms will not be able to reach the uterus, hence contraceptical effect is obtained.

### EXAMPLE 1

### Basic Type of Sperm-Blocking Agent Composition

Table I indicates the components of the composition and the units are weight percentage based on the total weight of the composition.

TABLE I
Components of the Basic Type Sperm-Blocking Agent

| | | |
|---|---|---|
| Gum | Carboxyethly cellulose | 3.8% |
| | HPMC | 0.1% |
| | MC | 0.15% |
| | High methoxyl pectin | 0.15% |
| | Sodium aliginate | 0.2% |
| | Xanthan gum | 0.1% |
| | Konjac gum | 0.1% |
| Gum Additive | gelatin | 0.1% |
| Lubricant | Glycerin | 3% |
| | High molecular weight polyethane | 2% |
| | oxide polymer | |
| | Polyethene glycol with molecular | 0.2% |
| | weight higher than 200 | |
| | Polypropylene glycol with molecular | 0.1% |
| | weight higher than 200 | |
| Lubricant Additive | Polymer of arylamide and dimethyl | 3% |
| | diallyl ammonium salt monomer | |
| Bacteriacide | methylphthalates | 0.15% |
| | Ethyl phthalates | 0.05% |
| Salt Additive | Sodium Chloride | 0.73% |
| Aqueous solution | Deionized water | 86.07% |

### EXAMPLE II

### Delayed-Ejection Type Sperm-Blocking Agent Composition

Table II indicates the components of the composition and the units are weight percentage based on the total weight of the composition. Bateriacides are used to increase the preservation period of the composition.

TABLE II
Delayed Ejection Type of Sperm-Blocking Agent Composition

| | | |
|---|---|---|
| Gum | Carboxyethly cellulose | 3.8% |
| | HPMC | 0.1% |
| | MC | 0.15% |
| | High methoxyl pectin | 0.15% |
| | Sodium alginate | 0.2% |
| | β-cyclodextrin amylase | 2.5% |
| | Xanthan gum | 0.1% |
| | Aloe Vera gel | 0.15% |
| | Locust bean gum | 0.1% |
| | curdlan | 0.1% |
| | Tara gum | 0.1% |
| Gum additive | gelatin | 0.1% |
| lubricant | glycerin | 3% |
| | High molecular polyethane oxide polymer | 2% |
| | Polyethene glycol with molecular weight | 0.2% |
| | higher than 200 | |
| | Polypropylene glycol with molecular | 0.1% |
| | weight higher than 200 | |
| Lubricant additive | Polymer of arylamide and dimethyl diallyl | 5% |
| | ammonium salt monomer | |
| bactericides | methylphthalates | 0.15% |
| | ethylphthalates | 0.05% |
| Salt additive | Sodium chloride | 0.68% |
| Aqueous solution | Deionized water | 81.27% |

### EXAMPLE III

### Frequent Sexual Intercourse Type Sperm-Blocking Agent Composition

Table III indicates the components of the composition and the units are weight percentage based on the total weight of the composition.

TABLE III
Frequent Sexual Intercourse Type Sperm-Blocking Agent
Composition.

| | | |
|---|---|---|
| Gum | Carboxyethly cellulose | 4.7% |
| | HPMC | 0.1% |
| | MC | 0.15% |
| | High methoxyl pectin | 0.15% |
| | Sodium alginate | 0.2% |
| | xantham | 0.1% |
| lubricant | glycerin | 1.2% |
| | High molecular polyethane oxide polymer | 2% |
| | Polyethene glycol with molecular weight | 0.2% |
| | higher than 200 | |
| | Polypropylene glycol with molecular | 0.1% |
| | weight higher than 200 | |
| Lubricant additive | Polymer of arylamide and dimethyl diallyl | 3% |
| | ammonium salt monomer | |
| bactericides | methylphthalates | 0.15% |
| | ethylphthalates | 0.05% |
| Salt additive | Sodium chloride | 0.75% |
| Aqueous solution | Deionized water | 87.15% |

The results of the product of the present invention mixed with sperm are shown below:

Experiment I: the reaction after mixing the sperm-blocking agent composition with sperm

5 cc of the sperm-blocking agent composition was coated onto a glass carrier with a thickness of 1 mm. 5 cc of sperm was dropped onto the glass carrier and was observed under a microscope with respect to the movement of sperm. Water contents of the sperm were absorbed and the viscosity increased. Thus, movement of sperm was difficult and subsequently, sperm stopped movement.

Experiment II: Observation of sperm on the reaction after mixing sperm-blocking agent composition with fluid secure from vagina 5 cc of the sperm-blocking agent composition was coated onto a glass carrier with a thickness of 1 mm. 5 cc of sperm together with equivalent saline (simulated fluid from vagina) was dropped onto the glass carrier. Observe the above under a microscope on the movement of sperm. Water from the sperm and the saline were absorbed and the viscosity increased. The sperms could not move easily. Subsequently, sperms stopped.

In view of the above experiments, the composition of the present invention restricts the movement of sperms and therefore sperm in the uterus will not move, and the composition provides a contraceptive effect. Further, the high viscosity gel blocks the cervicle and therefore, it further provides an effective contraceptive composition.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A sperm-blocking agent composition with contraceptive effect comprising:
an aqueous solvent
a gum which is a polysaccharide having about 0.3 to 10 wt % of the composition, wherein the polysaccharide is selected from the group consisting of celluloses and their derivatives, hemi-cellulose, fiber, starches and their derivatives, polydextrans and their derivatives, pectins and their derivatives, seaweed polysaccharids, their salts and derivatives and natural gums; and
a lubricant being alcohols having about 0.5 to wt % of the composition, wherein the alcohol is at least one selected from polyethene glycol having a molecular weight of over 200, polypropylene glycol having a molecular weight poly(ethylene oxide) polymers and glycerin;
wherein the viscosity of the composition is above 9000 cst.

2. The sperm-blocking agent of claim 1, wherein the aqueous solvent is deionized pure water.

3. The sperm-blocking agent of claim 2, further comprising a lubricating additive which is a polymer of arylamide and dimethyl diallyl ammonium salt monomer.

4. The sperm-blocking agent of claim 3, further comprising a gum additive, which is a gelatin.

5. The sperm-blocking agent of claim 4, further comprising a salt additive, which is sodium chloride.

6. The sperm-blocking agent of claim 5, wherein the celluloses and their derivatives are selected from the group consisting of carboxymethyl celluloses, and their derivatives, carboxyethyl celloloses and their derivatives, hydroxypropyl methyl celluloses (HPMC), and their derivatives, methyl celluloses (MC), and their derivatives, hydroxypropyl cellulose (HPC), and their derivatives, hydroxyethyl celluloses (HEC) and their derivatives, methyl hydroxyethyl celluloses (MHEC) and their derivatives, hydroxyl propyl methyl cellulose phthalates (HPMCP), and their derivatives.

7. The sperm-blocking agent of claim 6, wherein the starches and their derivatives are at least one selected from the group consisting of:
α-cyclodextrin amylases and their derivatives, β-cyclodextrin amylases and their derivatives, δ-cyclodextrin amylases and their derivatives, ζ-cyclodextrin amylases and their derivatives, ethylamide from starches and ?, dially ether from reaction of starches and epoxy methane, and reactant of starches and α-octyl butyldi-ester.

8. The sperm-blocking agent of claim 7, wherein the pectins and their derivaties is at least one selected from the group consisting of L-Rhamnose, high methoxyl pectin and low methoxyl pectin, amylopectine or alkyl pectin having a main chain added with a long chain.

9. The sperm-blocking agent of claim 8, wherein seaweed polysaccharids, their salts and the derivatives are selected from the group consisting of pheophycase, agarose, 1-carageenan, sodium alginate and cara gum

10. The sperm-blocking agent of claim 9, wherein the natural gum is selected from the group consisting of xanthan gum, konjac gum, agar, Flaxseed gum, arabic gum, guar gum, aloe vera gel, gellan gum, locust bean gum, curdlan gum, Tara gum, guar gum, Tragant gum, Tamarind gum, Karaya gum, and Furcellaran.

11. The sperm-blocking agent of claim 1, wherein the aqueous solvent is deionized pure water, and the gum includes carboxyethyl cellulose, and the lubricant includes glycerin and polyethylene oxide polymers of high molecular weight.

12. The sperm-blocking agent of claim 11, wherein further comprises of a lubricant additive, and a salt additive, and the lubricant additive includes
a polymer of arylamide and dimethyl diallyl ammonium salt monomer and the salt additive includes sodium chloride.

13. The sperm-blocking agent of claim 12, wherein the gum further comprises HPMC, MC, high methoxyl pectin and sodium alginate.

14. The sperm-blocking agent of claim 1, wherein the lubricant further comprises polyethene glycol with molecular weight of over 200, and polypropylene glycol with molecular weight of over 200.

15. The sperm-blocking agent of claim 1, wherein further comprises a gum additive which is a glycerin.

16. The sperm-blocking agent of claim 1, wherein the gum additive further comprises a xanthen gum and konjac gum.

17. The sperm-blocking agent of claim 16, wherein the composition includes
3.8%-carboxyethyl cellulose;
0.1%-HPMC;
0.15%-MC;
0.15%-high methoxyl pectin;
0.2%-sodium aliginate;
0.1%-xanthan gum;
0.1%-konjac gum;
0.1%-gelatin;
3%-glycerin;
2%-High molecular weight polyethane oxide polymer;
0.2%-Polyethene glycol with molecular weight higher than 200;
0.1%-Polypropylene glycol with molecular weight higher than 200;
3%-Polymer of arylamide and dimethyl diallyl ammonium salt monomer;
0.73%-sodium chloride; and
86.07% deionized pure water, and the components are in weight % of the composition.

18. The sperm-blocking agent of claim 15, wherein the gum the additive further comprises
α-cyclodextrin amylases, xanthan gum, aloe vera gum, locust bean gum, curdlan gum, Tara gum.

19. The sperm-blocking agent of claim 18, wherein the composition comprises,
3.8%-carboxyethyl cellulose;
0.1%-HPMC;
0.15%-MC;
0.15%- high methoxyl pectin;
0.2%,-Sodium alginate;
2.5%-β-cyclodextrin amylase;
0.1%-xanthan gum;
1.5%-Aloe Vera gel;
0.1%-Locust bean gum;
0.1%-curdlan;
0.1%-Tara gum;
0.1%-gelatin;
3%-glycerin;
2%-High molecular polyethane oxide polymer;
0.2%-Polyethene glycol with molecular weight higher than 200;
0.1%-Polypropylene glycol with molecular weight higher than 200;
5%-Polymer of arylamide and dimethyl diallyl ammonium salt monomer
0.68%-Sodium chloride, and
81.27%-Deionized water

20. The sperm-blocking agent of claim 14, wherein the gum further comprises xanthan gum.

21. The sperm-blocking agent of claim 20, wherein the composition comprises:
4.7%-carboxyethyl cellulose (CEC);
0.1%-HPMC;
0.15%-MC;
0.15%-high methoxyl pectin;
0.2%-sodum alginate;
0.1%-xanthan;
1.2%-glycerin;
2%-high molecular weight polyethene oxide polymer;
0.2%-polyethylene glycol of high molecular weight higher than 200;
0.1%-polypropylene glycol of high molecular weight higher than 200;
3%-Polymer of arylamide and dimethyl diallyl ammonium salt monomer;
0.75% sodium chloride; and
87.15% deionized pure water, and the components are in weight % of the composition.
